(19) United States
(12) Patent Application Publication
(10) Pub. No.: US 2025/0360179 A1
(43) Pub. Date: Nov. 27, 2025

(54) PEPTIDE-IONIC LIQUID CONJUGATE FOR THE PREVENTION AND/OR TREATMENT OF SKIN DISORDERS

(71) Applicants: UNIVERSIDADE DO PORTO, PORTO (PT); REQUIMTE - REDE DE QUÍMICA E TECNOLOGIA, PORTO (PT); INSTITUTO POLITÉCNICO DO PORTO, PORTO (PT)

(72) Inventors: Paula Alexandra DE CARVALHO GOMES, PORTO (PT); Cátia Andreia SILVA TEIXEIRA, PORTO (PT); Ana Sofia MARTINS GOMES, PORTO (PT); Alberta Paula LOBO MACHADO GAMEIRO DOS SANTOS, PORTO (PT); Nuno Filipe DA CRUZ BATISTA MATEUS, PORTO (PT); Iva Luzia REIS FERNANDES, PORTO (PT); Lucinda Janete DA SILVA BESSA, CHARNECA DA CAPARICA (PT); Ricardo João VIEIRA FERRAZ, Porto (PT); Maria Cristina PRUDÊNCIO SOARES, Porto (PT)

(21) Appl. No.: 18/876,323

(22) PCT Filed: Jun. 23, 2023
(86) PCT No.: PCT/IB2023/056516

§ 371 Date: Dec. 18, 2024

(30) Foreign Application Priority Data
Jun. 24, 2022 (PT) 118065

(51) Int. Cl. A61K 38/06; A61K 8/64; A61K 38/07; A61K 38/08; A61K 47/54; A61P 17/00; A61P 31/04; A61P 31/10; A61Q 19/08

(52) U.S. Cl. CPC A61K 38/06; A61K 8/64; A61K 38/07; A61K 38/08; A61K 47/541; A61P 17/00; A61P 31/04; A61P 31/10; A61Q 19/08

(57) ABSTRACT

A peptide-ionic liquid conjugate for the prevention and/or treatment of skin disorders. The peptide-ionic liquid conjugate comprises a cosmeceutical peptide comprising up to 10 amino acids and at least one ionic liquid comprising saturated or unsaturated hydrocarbon chain substituents from C1 to C18. The peptide-ionic liquid conjugates herein disclosed are suitable against skin disorders, such as, e.g., melanoma, non-melanoma skin cancers, diabetic foot ulcers, venous leg ulcers, pressure ulcers, acne, candidiasis, cellulitis, dermatophytoses, erysipelas, folliculitis, impetigo, psoriasis, rosacea, or eczema (atopic dermatitis), since they present potent activity against either antibiotic-susceptible strains or multidrug resistant clinical isolates of both Gram-positive and Gram-negative, a bactericidal type of action, anti-inflammatory and immunomodulatory effects, and collagenesis-inducing effects. Further disclosing a topical composition comprising the peptide-ionic liquid conjugates and uses thereof.

### TECHNICAL FIELD

This application relates to a peptide-ionic liquid (PIL) conjugate for the prevention and/or treatment of skin disorders, a topical composition comprising the same and uses thereof.

### BACKGROUND ART

Ionic Liquids (ILs), though mostly known for their potential role as “green solvents”, are becoming increasingly attractive as easily customizable and tunable organic salts for diverse specific purposes (task-specific ILs). There are infinite possibilities when combining organic cations with organic or inorganic anions, enabling production of ILs with diverse structural, physical and chemical properties [1] that can be adapted to the demands of areas as diverse as material sciences [2], biotechnology [3], or biomedicine [4]. Moreover, by making use of bioactive ions, ILs displaying relevant biological activities [5] can be produced, for instance, as anticancer [6], antimalarial [7], and antimicrobial agents [8]. ILS showing broad-spectrum activity against both Gram-negative and Gram-positive bacteria and antibiofilm activity have been reported [9]; as such, ILs are emerging as appealing alternatives to counteract antimicrobial resistance, while the world is running out of effective antibiotics, especially against Gram-negative bacteria. Furthermore, many ILs have gained attention as dermal permeation enhancers [10], making them particularly attractive for topical applications. ILs presenting both antimicrobial activity and dermal permeation enhancement can be quite helpful to treat skin disorders including infected injuries, as recently demonstrated in an in vivo biofilm-infected wound assay, where an IL was able to kill 95% of the bacteria [11].

The prevalence of diabetes, peripheral vascular disease, or conditions forcing patients to be bedridden, among others, is increasing alongside with life expectancy, and is often associated with problems like diabetic foot ulcers (DFU) and other skin disorders, such as complicated skin and soft tissue infections (cSSTI) [12]. Despite cSSTI are mostly caused by multidrug resistant (MDR) bacteria from the ESKAPE (Enterococcus faecium, Staphylococcus aureus, Klebsiella pneumoniae, Acinetobacter baumannii, Pseudomonas aeruginosa, and Enterobacter spp.) group, a high prevalence of fungi in chronic wounds has also been reported, and linked to both healing time, and formation of mixed biofilms with bacteria; Cladosporidium spp. and Candida spp., mainly C. albicans and C. parapsilosis, were described as the most prevalent fungi in cSSTI [13,14]. The burden of cSSTI increased during the COVID-19 pandemic due to reluctance in exposing elderly patients to healthcare facilities [15] and is exacerbated by both the (i) dissemination of MDR pathogens, and (ii) installation of polymicrobial biofilms that are refractory to both current antibiotics and the immune system, and delay or impair healing.

In the specific case of cSSTI, treatment requires debridement or incision and drainage, complemented with antibiotic therapy. The guidelines for cSSTI treatment the recommend administration of systemic antibiotics that are effective against methicillin-resistant Staphylococcus aureus (MRSA) strains, which are among MDR pathogens that prevail in health care facilities, followed by an antibiotic therapy program based on the culture assessments for a definitive treatment [16]. When this fails, amputation emerges as the last resource to avoid life-threatening sepsis.

Resistance to the available antibiotics is rapidly increasing and currently widespread to many different species of Gram-positive and Gram-negative bacteria. Likewise, fungal infections are becoming more difficult to treat due to the development and spread of resistance among pathogenic fungi.

Peptide-based antimicrobials, like the cyclic lipopeptide daptomycin, offer clinicians an alternative to tackle MDR bacteria in hospital settings, but daptomycin is exclusively active against Gram-positive species, including MRSA (17). However, most cSSTI are of polymicrobial nature, also involving Gram-negative bacteria and fungi, meaning that new options are urgently needed to cope with the emerging post-antibiotic era (18). Fungal colonization of non-healing wounds has been traditionally neglected (14), but Kalan et al. have highlighted the importance of identifying not only bacterial, but also fungal species involved in cSSTI (14), namely, Cladosporidium spp. and Candida spp., including C. albicans and C. parapsilosis (19).

The healing process in skin disorders is often delayed or even impaired due to age- and/or disease-related pathophysiological traits, such as high oxidative stress, neurophaty, angiopathy, poor tissue oxygenation, chronic and inflammation, poor immune response; this scenario is further aggravated by installation of microbial pathogens that promptly form biofilms in the wound bed, and which are refractory to the action of most current antibiotics (20). In such cases, effective treatment of skin lesions must, most of the times, both quell infection and promote fast and correct healing. In this regard, collagen, as a structural protein from the extracellular matrix (ECM), plays an important role in key steps of wound healing and closure (21). Because collagen is an endogenous protein, it is regarded as a desirable component for the development of biocompatible and biodegradable wound dressings/biomaterials (22). Along with collagen itself, collagen-derived/inspired peptides, such as cryptic collagen peptides (23), have been also considered as potential promoters of cell migration and proliferation, capable of inducing fibroblasts to produce new collagen, and consequently promoting faster wound healing (24). This motivated the development of the so-called cosmeceutical peptides (CP), and derivatives thereof, for topical application in the treatment of skin disorders, acting by promoting faster skin regeneration. CP are small peptide fragments that result from proteolysis of ECM macromolecules like collagen or elastin, with diverse potential biomedical applications, including in cosmetics (25, 26). For instance, “pentapeptide-4” (or PP4, with amino acid sequence KTTKS) is a widely studied CP that derives from type I human collagen and is the smallest peptide sequence known to retain a potent ability to stimulate ECM (collagen and fibronectin) production (27, 28). The N-palmitoylated form of KTTKS, known as “palmitoyl pentapeptide-4” or Matrixyl®, is used in the cosmetics industry due to its ability to cause a skin-rejuvenation/anti-wrinkle effect, probably associated to a collagenesis-inducing action (29, 30).

Noteworthy, as previously mentioned, a sensible choice of peptide and ionic liquid building blocks to be conjugated enables the development of a range of products tailored for their intended specific topical use, covering from severe non-healing skin ulcerations (diabetic foot ulcers, pressure ulcers, venous leg ulcers, and alike) to usually milder bacterial or fungal skin infections (acne, atopic dermatitis, candidiasis, cellulitis, dermatophytoses, erysipelas, folliculitis, impetigo, rosacea, and others), but also post-surgical wounds, common injuries or Moreover, burns. considering that peptide-based antimicrobials have been widely associated to other activities such as immunomodulatory, antioxidant, anti-aging and anti-tumoral effects, likely find application also in the topical our products will treatment of auto-immune/inflammatory, aging and cancer skin disorders, such as psoriasis, atopic dermatitis, cutaneous lupus erythematosus, impetigo, skin aging, and melanoma, either in absence or presence of secondary infection.

The potential of PP4 to promote skin regeneration in the context of skin lesions has recently attracted attention. Thus, given that this CP is devoid of antimicrobial action, when the KTTKS sequence is conjugated to an antimicrobial peptide (AMP), this results in a chimeric peptide displaying (i) antibacterial activity against reference and MDR bacteria from clinical isolates; (ii) antibiofilm action; and (iii) a collagenesis-inducing effect comparable to that of Matrixyl® (32).

Further N-terminal modification of the aforementioned chimeric peptide with an imidazolium-based ionic liquid afforded an equally potent antimicrobial construct with increased stability toward enzyme-mediated modification (31). Indeed, IL are becoming quite attractive for biomedical applications, given their unique physicochemical characteristics, low cost, and high structural diversity that enables the synthesis of a wide panoply of different IL which can be easily tuned to meet specific requirements, including broad spectrum activity against bacteria (4) and fungi (33). Recently, alkylimidazolium-based IL have been proposed as an alternative antibacterial treatment for cSSTI focusing on Gram-positive pathogens (34). In addition, several IL have been found to improve the skin permeation of drugs (10, 35, 36, 37), including ceftazidime, an antibiotic possessing poor water solubility and low skin permeation (11).

Document “Clicking” an Ionic Liquid to a Potent Antimicrobial Peptide: On the Route towards Improved Stability (31) describes an analogue of the peptide 3.1-PP4 (composed of the antimicrobial peptide 3.1 and the cosmeceutical pentapeptide-4) conjugated with the ionic liquid methyl imidazolium at the N-terminus (MeIm-3.1-PP4) and its biological application as antibacterial, antibiofilm and antifungal agent. This conjugate retained the unmodified parent chimeric peptide's activity (3.1-PP4) against multidrug-resistant clinical isolates of Gram-negative bacteria, and antibiofilm action on a resistant clinical isolate of Klebsiella pneumoniae, while exhibiting much improved stability towards tyrosinase-mediated modifications.

Document “Disclosure of a Promising Lead to Tackle Complicated Skin and Skin Structure Infections: Antimicrobial and Antibiofilm Actions of Peptide PP4-3.1” (38) describes an analogue of the peptide PP4-3.1 (composed of the cosmeceutical pentapeptide-4 and the antimicrobial peptide 3.1) conjugated with the ionic liquid methyl imidazolium at the N-terminus (MeIm-PP4-3.1) and its biological application as antibacterial, antibiofilm and antifungal agent. Although the conjugated peptide presented activity in all the biological assays, the unmodified parent chimeric peptide (PP4-3.1) stood out for its potent activity against Gram-positive and Gram-negative bacteria, including against MDR clinical isolates, and against three clinically relevant species of Candida fungi, with an overall performance superior to that of MeIm-PP4-3.1.

Both documents describe peptide analogues with antibacterial, antibiofilm and antifungal activity in vitro, exhibiting N-terminal modification of chimeric peptides combining a cosmeceutical and an antimicrobial sequence. Nonetheless, these documents do not describe peptide analogues comprising a quaternary imidazolium moiety with long hydrocarbon chain substituents and do not provide any demonstration of a significant collagen biosynthesis-inducing activity of the peptide derivatives therein reported. Also, the peptide analogue sequences reported in the two documents comprise long and highly cationic host defence peptide sequences, given their well-known direct antimicrobial action. Yet, many host defence peptides lack the fast and potent skin regeneration ability that can be afforded by cosmeceutical peptides, and most are too long (with over 10 amino acids) for an efficient dermal and transdermal delivery (39) and for cost-effective production at industrial scale. Moreover, host defence peptide-resistant strains of the most prevalent bacterial pathogen in infected skin lesions, Staphylococcus aureus, have been reported to possess an increased membrane surface charge, which lowers the efficiency of the initial peptide-bacterial electrostatic interaction that precedes the peptide's bactericidal action by bacterial membrane destabilization (40); this means that the formerly developed constructs based on host defence peptides might trigger selection of resistant microbial strains. In turn, the new antimicrobial constructs derive from small CP and not from highly cationic host defence peptides, hence are less likely to induce resistance.

Gomes et al. in ‘The Emerging Role of Ionic Liquid-Based Approaches for Enhanced Skin Permeation of Bioactive Molecules: A Snapshot of the Past Couple of Year’, Int. J. Mol. Sci., 2021, 22, 11991, discloses a review of ionic liquids as dermal permeation enhancers, but it does not disclose technical information related to the present invention.

Bergfeld et al. in ‘Safety Assessment of Tripeptide-1, Hexapeptide-12, their Metal Salts and Acyl Fatty Derivatives, and Palmitoyl Tetrapeptide-7 as Used in Cosmetics’, Cosmetic Ingredient Review 2014, is a study in the safety of using peptides in cosmetics but does not address antimicrobial action of the peptides or discloses technical information related to the present invention.

Gomes et al. in ‘“Clicking” an Ionic Liquid to a Potent Antimicrobial Peptide: On the Route towards Improved Stability’, International Journal of Molecular Sciences, vol. 21, n.° 17, 2020, does not disclose a cosmeceutical peptide with up to 10 amino acids, it is a 16-aminoacid antimicrobial peptide that comprises both antimicrobial and cosmeceutical motifs within its sequence. The present invention differs from this document because the ionic liquid is covalently attached to the cosmeceutical is peptide, which devoid of antimicrobial action per se, without resorting to antimicrobial peptide motifs.

The present invention refers only to small (up to 10 amino acids) CP already amply used (and patented) as active ingredients in cosmetics, for cosmetic applications, but in no case as topical antimicrobials, given their lack of intrinsic antimicrobial action. Relevantly, the features of PIL conjugates to which the present invention refers are not simply a mere sum of the intrinsic properties of each of the two individual building blocks, as simply mixing them (instead of linking them through chemical conjugation) delivers mixtures with bacteriostatic rather than bactericidal action. In addition, the chemical modifications of peptide analogues from the two documents mentioned above (31, 38) are only carried out at their N-termini, while the PIL conjugates in the present invention have one or multiple IL building blocks attached to either the N-terminus or amino acid side chains (e.g., azido-lysine side chains) or both. Insertion on amino acid side chains for chemical conjugation of IL to peptides has never been reported so far.

### SUMMARY

The present invention related to a peptide-ionic liquid conjugate for the prevention and/or treatment of skin disorders, wherein the peptide-ionic liquid conjugate comprises a cosmeceutical peptide comprising up to 10 amino acids and at least one ionic liquid comprising saturated or unsaturated hydrocarbon chain substituents from C1 to C18.

In one embodiment the cosmeceutical peptide is selected from a cosmeceutical peptide selected from any of the sequences SEQ. ID NO 1 to 20.

In one embodiment the at least one ionic liquid is selected from a pyridinium, an imidazolium, a phosphonium, or a cholinium ionic liquid.

In one embodiment the conjugation between the cosmeceutical peptide and at least one ionic liquid occurs on the N-terminus of an amino acid of the cosmeceutical peptide.

In one embodiment the conjugation between the cosmeceutical peptide and at least one ionic liquid occurs on a side chain of an amino acid of the cosmeceutical peptide.

In one embodiment the conjugation between the cosmeceutical peptide and the ionic liquids occurs on the N-terminus of an amino acid and on a side chain of an amino acid of the cosmeceutical peptide.

The present application also relates to a topical composition comprising at least one peptide-ionic liquid conjugate type.

In one embodiment the topical composition further comprises nanoparticles, solid lipid nanoparticles, nanostructured lipid carriers including liposomes or polymeric nanoparticles, and/or at least one hydrogel including polysaccharide-based hydrogels or poly(lactic-co-glycolic acid)-based hydrogels.

In one embodiment the topical composition is for use in the prevention and/or treatment of skin disorders.

In one embodiment the skin disorders are severe non-healing skin ulcerations such as diabetic foot ulcers, pressure ulcers, venous leg ulcers; milder bacterial or fungal skin infections such as acne, atopic dermatitis, candidiasis, cellulitis, dermatophytoses, erysipelas, folliculitis, impetigo, rosacea; auto-immune/inflammatory disorders, aging and cancer skin disorders, such as melanoma, non-melanoma skin cancers, psoriasis, cutaneous lupus erythematosus, impetigo, skin aging, and melanoma, either in absence or presence of secondary infection, treatment of post-surgical wounds, common injuries or burns.

### GENERAL DESCRIPTION

This application relates to a peptide-ionic liquid (PIL) conjugate suitable for the prevention and/or treatment of skin disorders.

The peptide-ionic liquid conjugate described herein is a novel type of prospective Active Pharmaceutical Ingredient (API) suitable for topical compositions to prevent and/or treat disorders of the skin.

The present invention relates to the conjugate of a cosmeceutical peptide (CP) comprising up to 10 amino acids, with at least one ionic liquid, forming a PIL conjugate.

As an example, the direct coupling of alkylimidazolium-based IL to the non-antimicrobial pentapeptide-4 (PP4) with sequence SEQ. ID 1 (a cosmeceutical peptide with up to 10 amino acids: KTTKS) was studied to understand if it would afford a new type of peptide-based construct displaying collagenesis-inducing and antimicrobial action, despite not harboring an antimicrobial peptide (AMP) motif. To this end, three different alkylimidazole-based ILs were chemically modified to introduce the alkyne moiety required for subsequent to coupling different azide derivatives of PP4. Seven different IL-PP4 conjugates were produced, and their antibacterial, antifungal, and collagenesis-inducing properties studied, as herein reported and discussed.

These conjugates present potent activity against either antibiotic-susceptible strains or multidrug resistant clinical isolates of both Gram-positive and Gram-negative bacterial species belonging to the so-called “ESKAPE” group of pathogens. Noteworthy, their antibacterial activity is preserved in simulated wound fluid, which anticipates an effective action in the setting of a real wound bed. Moreover, their collagenesis-inducing effects in vitro are comparable to or stronger than those of Matrixyl®. Altogether, IL-PP4 exert a triple antibacterial, antifungal, and collagenesis-inducing action in vitro. These findings provide solid grounds to advance IL-PP4 conjugates as promising leads for future development of topical prevention and/or treatment for skin disorders.

Further studies are envisaged to incorporate PIL conjugates into suitable nanoformulations, to reduce toxicity, improve resistance to proteolytic degradation, and retain the active pharmaceutical ingredient at the intended site of action (wound bed, lesion area).

The major advantages of the PIL conjugates of the present invention are:
1. the fast skin regeneration ability and safety for use on human skin conveyed by the CP, which are already used in cosmetics for advanced skin care;
2. the small size of the CP (up to 10 amino acids), favouring their cost-effective production and skin permeation ability, without the need for using non-trivial/expensive dermal and transdermal delivery enhancement methods (e.g., electroporation, iontophoresis, sonophoresis, microneedles, etc.);
3. the fact that CP are already produced at industrial scale according to current good manufacture practices (cGMP);
4. the widely reported antimicrobial and skin permeation ability of IL, namely but not limited to, imidazolium and pyridinium-based; for instance, cetylpyridinium chloride is an antiseptic IL used in mouthwashes, toothpastes, and throat/nasal sprays that combines microbicidal action with dermal permeation ability (41);
5. the possibility of using a fast and straightforward chemoselective conjugation between the CP and the IL building blocks by click chemistry approaches, namely but not limited to, copper(I)-catalysed azide-alkyne coupling (CuAAC);
6. the likelihood of delivering new products that could be fine-tuned not only to reflect the sum of the properties of each of its building blocks, but also to display new properties (e. g., immunomodulatory), hence expanding its prospective range of therapeutic (and cosmetic) applications beyond management of skin infections and wound healing (i.e., topical use on autoimmune skin disorders like psoriasis or eczema).

### BRIEF DESCRIPTION OF DRAWINGS

For easier understanding of this application, figures are attached in the annex that represent the preferred forms of implementation which nevertheless are not intended to limit the technique disclosed herein.

FIG. 1—Route to the target IL-PP4 conjugates. (A) Synthesis of the alkyne derivatives of the IL: (i) 1.1 eq of C₁₆Im, C₁₄Im or MeIm and 1.0 eq of propargyl bromide (80% in toluene), 40° C., 24 h. (B) Synthesis of the azide derivatives of KTTKS and their coupling to the alkynyl-IL via CuAAC: (ii) 5 eq of Fmoc-protected amino acid, 10 eq of N-ethyl-N,N-diisopropylamine (DIEA) and 5 eq of O-(benzotriazol-1-yl)-N,N,N′,N′-tetramethyluronium hexafluorophosphate (HBTU) in N,N-dimethylformamide (DMF), 1 h, room temperature (r.t.); (iii) 20% piperidine in DMF, 15 min, r.t.; (iv) 5 eq of azido acetic, 10 eq of DIEA, 5 eq of HBTU, 1 h, r.t.; (v) 10 eq of DIEA, 10 eq of 2,6-lutidine, 1 eq of copper(I) bromide, 1 eq of sodium L-ascorbate and 1 eq of either Pr-MeIm, Pr-C₁₆Im, or Pr-C₁₄Im, in DMF: acetonitrile (ACN) (3:1 v/V), 24 h, (vi) r.t.; trifluoroacetic acid (TFA)/triisopropylsilane (TIS) distilled water (95:25:2.5 v/v/v).

FIG. 2—Structure of IL-PP4 conjugates and corresponding molecular weight (in Da). The amino acids are represented in single letter code as defined by the IUPAC-IUBMB guidelines on nomenclature and symbolism for amino acids and peptides; exception is made to the amino acid residues whose side chain was coupled to ionic liquids via click chemistry, in which case the full modified structure is shown.

FIG. 3—Collagen synthesis by Human Dermal Fibroblasts (HDF), in presence of C₁₆-KTTKS-OH (Matrixyl®), KTTK (C₁₆Im)S and C₁₆Im-KTTKS at 5 μM, using the Sircol™ Kit. Data are presented as mean±SEM (3 independent experiments in triplicates) expressed in collagen amount (% of control); *p<0.05, **p<0.01.

### DESCRIPTION OF EMBODIMENTS

NOW, preferred embodiments of the present application will be described in detail with reference to the annexed drawings. However, they are not intended to limit the scope of this application.

The present invention relates to a peptide-ionic liquid conjugate of a cosmeceutical peptide (CP) comprising up to 10 amino acids and at least one ionic liquid, forming an PIL conjugate.

In one embodiment, the cosmeceutical peptide is selected from, but not limited to, PP4 (SEQ. ID 1), KVK, PKEK (SEQ. ID 2), GEKG (SEQ. ID 3), GHK, copper tripeptide-1, manganese tripeptide-1, palmitoyl tripeptide-1, tripeptide-5, palmitoyl tripeptide-5, tetrapeptide-3 (SEQ. ID 4), pentapeptide-3 (SEQ. ID 5), pentapeptide-18 (SEQ. ID 6), hexapeptide-11 ID (SEQ. 7), acetyl hexapeptide-3 (SEQ. ID 8), hexapeptide-10 (SEQ. ID 9), hexapeptide-12 (SEQ. ID 10), palmitoyl hexapeptide-12, acetyl octapeptide-1 (SEQ. ID 11), SA1-III (SEQ. ID 12), lipospondin.

In one embodiment the at least one ionic liquid is selected from, but not limited to, any ionic liquid comprising a saturated or unsaturated hydrocarbon chain substituent from C₁ to C₁₈. In one embodiment the ionic liquid is selected from diverse ionic liquid families, namely but not limited to, a pyridinium (e.g., cetylpyridinium), an imidazolium (e.g., 1-alkyl-3-methylimidazolium), a phosphonium (e.g. trihexyltetradecylphosphonium), or an cholinium ionic liquid.

The conjugation between the cosmeceutical peptide and at least one ionic liquid occurs on the N-terminus of an amino acid and/or on a side chain of a specific amino acid in the peptide sequence, namely but not limited to, lysine, arginine, and histidine side chains.

In one embodiment, the PIL-conjugates are for use in the treatment and/or prevention of skin disorders, from severe non-healing skin ulcerations such as diabetic foot ulcers, pressure ulcers, venous leg ulcers, and alike, to usually milder bacterial or fungal skin infections such as acne, atopic dermatitis, candidiasis, cellulitis, dermatophytoses, erysipelas, folliculitis, impetigo, rosacea, and others, but also auto-immune/inflammatory disorders, aging and cancer skin disorders, such as melanoma, non-melanoma skin cancers, psoriasis, cutaneous lupus erythematosus, impetigo, skin aging, and melanoma, either in absence or presence of secondary infection. It may also find application in the treatment of post-surgical wounds, common injuries or burns.

The present application also relates to a topical composition comprising at least one type of PIL conjugate.

In one embodiment, the topical composition comprising the PIL-conjugate is for use in the treatment and/or prevention of skin disorders, from severe non-healing skin ulcerations such as diabetic foot ulcers, pressure ulcers, venous leg ulcers, and alike, to usually milder bacterial or fungal skin infections such as acne, atopic dermatitis, candidiasis, cellulitis, dermatophytoses, erysipelas, folliculitis, impetigo, and rosacea, others, but also auto-immune/inflammatory disorders, aging and cancer skin disorders, such as melanoma, non-melanoma skin cancers, psoriasis, cutaneous lupus erythematosus, impetigo, skin aging, and melanoma, either in absence or presence of secondary infection. It may also find application in the treatment of post-surgical wounds, common injuries or burns.

In one embodiment, the topical composition further comprises but not limited to, solid lipid nanoparticles, including, nanoparticles, nanostructured lipid carriers (liposomes or polymeric nanoparticles) and/or at least one hydrogel including, but not limited to, polysaccharide-based hydrogels or poly(lactic-co-glycolic acid)-based hydrogels.

It has been previously demonstrated that it is possible to produce a dual-action antimicrobial and collagenesis-inducing chimeric peptide, by combining the amino acid sequence of an AMP to that of the non-antimicrobial well-known cosmeceutical peptide PP4 (32). It was further shown that such potent antimicrobial activity was preserved when coupling an imidazolium IL to the N-terminus of the chimeric peptide via the CuAAC “click” approach, which conferred the peptide resistance to enzyme-mediated modification (31).

Based on these findings, the studies disclosed in the present application were to investigate if the dual antimicrobial and collagenesis-inducing was activity preserved by removing the AMP sequence and coupling at least one IL directly to the amino acid sequence of the cosmeceutical peptide PP4.

The findings disclosed in the present application strongly indicate that the direct conjugation between an IL and other cosmeceutical peptides comprising up to 10 amino acids will have the same properties observed in the examples herein disclosed.

In the experimental examples ahead the PP4 peptide will be mentioned by its amino acid sequence, i.e., KKTKS.

The antibacterial activity of the new constructs herein presented, IL-KKTKS, was assessed against reference bacterial strains and results obtained (shown in the Examples section) allowed to advance a couple of structure-activity relationships (SAR) on the (i) IL insertion site, as the covalent graft was at either the N-terminus or the Lys1/Lys4 side chains of the KTTKS sequence, and (ii) length of the alkyl substituent in the imidazole ring, which was varied between one (methyl or Me), fourteen (tetradecyl or C14), and sixteen (hexadecyl or C16) carbons. Hence, antibacterial activity (i) increased with the length of the alkyl substituents in the IL moiety [KTTK(C14Im)S versus KTTK(C16Im)S] and (ii) is depleted in all conjugates bearing the methyl-substituted imidazolium IL, regardless of other structural features.

Moreover, MIC values were also determined for the parent building blocks KTTKS and [C16 M1Im][Br], as well as for their noncovalent equimolar mixture, indicated as KTTKS:[C16 M1Im][Br] (1:1), so that the importance of covalent conjugation could be assessed. The noncovalent mixture KTTKS:[C16 M1Im][Br] (1:1) presented MIC values similar to those of [C16 M1Im][Br] alone, confirming that the IL building block is the main responsible for the activity observed for the mixture, as expected.

Interestingly, when comparing the MIC values of the noncovalent mixture KTTKS:[C16 M1Im][Br] (1:1) with those of the covalent conjugates KTTK(C16Im)S and C16Im-KTTKS, it is apparent that covalent conjugation is clearly beneficial for activity against Gram-negative bacteria, but not so much against Gram-positive bacteria. Still whereas the noncovalent mixture is bacteriostatic for Gram-positive species at MIC values, the covalent conjugates are bactericidal at these concentrations. These results indicate that the antibacterial activity of the covalent conjugates KTTK(C16Im)S and C16Im-KTTKS is not only modulated by the IL building block, but also by its conjugation to the peptide. Hence, biophysical studies will be performed to further explore the mechanism(s) of action of IL-KTTKS conjugates. The conjugates KTTK(C16Im)S and C16Im-KTTKS also showed a potent activity against MDR clinical isolates of Gram-positive and Gram-negative bacteria, being more active than the reference antibiotic ciprofloxacin.

This is a relevant finding, considering that the three clinical isolates tested refer to bacterial species belonging to the “ESKAPE” group of pathogens which encompasses life-threatening nosocomial pathogens, namely, Enterococcus faecium, Staphylococcus aureus, Klebsiella pneumoniae, Acinetobacter baumannii, Pseudomonas aeruginosa, and Enterobacter spp. The ability of “ESKAPE” pathogens to escape the action of currently available antibiotics is one of the major healthcare threats of our times, especially for Gram-negative bacteria, against which the world is running out of effective options.

Additionally, the conjugates showed to retain (C16Im-KTTKS) or slightly decrease (KTTK(C16Im)S) the antibacterial activity against S. aureus in simulated wound fluid (SWF). These preliminary observations using SWF, which mimics the wound exudate, are relevant as they are indicative that the IL-peptide conjugates, especially C16Im-KTTKS, can be more stable in the wound environment as compared to analogues where the peptide is not protected at the N-terminus.

The IL-KTTKS conjugates herein described have also shown interesting antifungal properties, especially against C. parapsilosis. This is one of the most common non-C. albicans species of Candida, which are regarded as important nosocomial pathogens of concern as they were reported to be involved in cases of sepsis and cSSTI. In this regard, the antifungal activity of the conjugates was assessed, and MIC values observed were as low as 2.4 and 2.7 μM against C. parapsilosis and 4.7 and 5.7 μM against C. albicans.

Still, as observed in antibacterial activity assays, the non-covalent mixture and the parent IL are more potent than the IL-KTTKS conjugates against Candida spp., which indicates that the antifungal activity of the covalent conjugates is not only modulated by the IL building block, but also by their conjugation to the peptide (devoid of antifungal activity). Therefore, further biophysical studies will be performed to shine some light into possible mechanism(s) of action of IL-KTTKS conjugates against Candida species.

The evaluation of the cytotoxicity of the IL-KTTKS conjugates is obviously important on its own to check for selectivity, but also due to the toxicity effects often associated to IL, depending on, e.g., cation alkyl chain length or specific ions used.

The parent IL [C16 M1Im][Br] and its covalent equimolar mixture with the peptide, KTTKS:[C16 M1Im][Br] (1:1), were significantly toxic against the human cell lines tested. Therefore, covalent conjugation of the IL to the peptide confers on one hand, antimicrobial activity to an otherwise peptide building block devoid of such activity and, on the other, reduced cytotoxicity as compared to the parent IL.

The conjugates C16Im-KTTKS and KTTK(C16Im)S were further assessed for their ability to induce collagen production by human dermal fibroblasts in vitro. The conjugates showed to be comparable to the reference cosmeceutical Matrixyl® and more active than the control. No significant difference between both conjugates were observed, indicating that changing the side chain of Lys4 did not affect the peptide's collagenesis-inducing behavior.

Altogether, the findings on the present examples are unprecedented as well as remarkable, as they provide confirmation by advancing a couple of C16Im-KTTKS and PIL conjugates, KTTK(C16Im)S, that possess antibacterial, antifungal, and collagenesis-inducing activity in vitro, the latter being actually comparable to that of the cosmeceutical ingredient Matrixyl® based on the KTTKS peptide. Further, the site of insertion of the IL does not significantly affect the overall in vitro properties of the conjugates [C16Im-KTTKS versus KTTK(C16Im)S], although N-terminal conjugation seems to better preserve the conjugates' antibacterial action in SWF and to improve the collagen synthesis by human dermal fibroblasts.

Further studies are envisaged to incorporate PIL into nanoformulations and/or hydrogels, which will reduce toxicity, improve resistance to proteolytic degradation, and retain the active pharmaceutical ingredient at the intended site of action (wound bed, lesion area). Moreover, since IL-KKTKS the conjugates will be applied in the treatment of cSSTI, which are mainly polymicrobial infections, the antimicrobial activity of IL-KTTKS on polymicrobial cultures will be further investigated. This will enable selection of best IL-KTTKS based formulations to advance for in vivo studies.

Considering that KTTKS and other small cosmeceutical peptides are already produced at industrial scale as ingredients for cosmetic products, these findings unveil the value of IL-CP conjugates as a promising start for future development of cost-effective topical formulations for the prevention and/or treatment of skin disorders, from mild to severe ones like cSSTI.

### EXAMPLES

### 1. Synthesis of the Target Conjugates

The route towards the target IL-KTTKS conjugates started by the synthesis of the alkyne-modified imidazolium IL (FIG. 1-A). 1-methyl-imidazole (Me-IM), 1-tetradecyl-imidazole (C14-Im) and 1-hexadecylimidazole (C16-Im) were reacted with propargyl bromide according to Hu et al. (FIG. 1-A) (42), to afford the three target imidazolium ILs, propargyl-MeIm (Pr-MeIm), propargyl-C₁₄Im (Pr-C₁₄Im) and propargyl-C₁₆Im (Pr-C₁₆Im). The structures of these ILs were confirmed by ¹H-NMR, ¹³C-NMR, and ESI-IT MS.

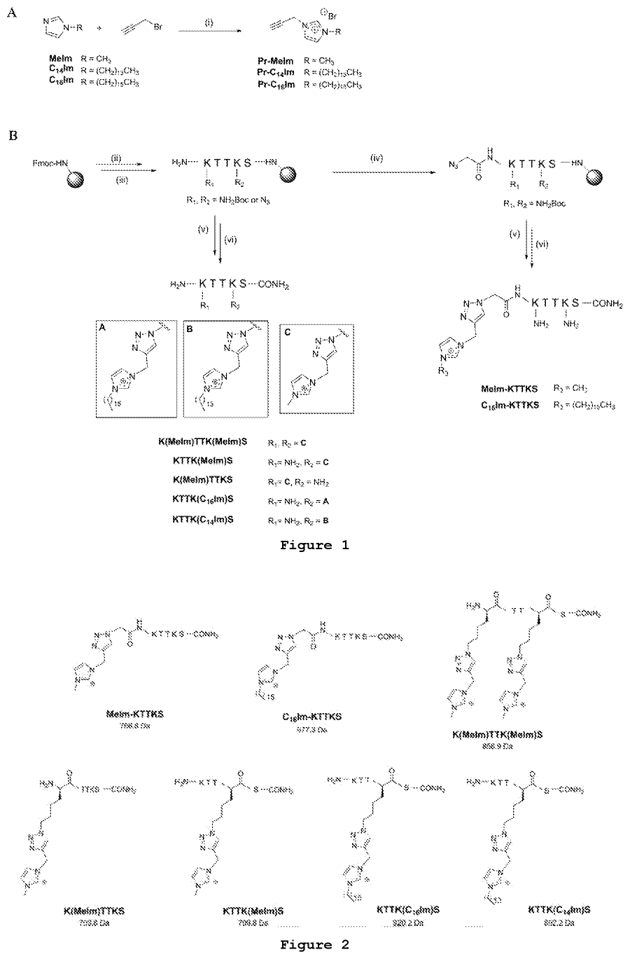

In parallel, conveniently modified derivatives of PP4 (amino acid sequence KTTKS) were produced by Solid Phase Peptide Synthesis (SPPS), to afford diverse final IL-KTTKS conjugates (FIG. 2) that differed in the: (a) propargyl-imidazolium building blocks used, (b) insertion site of the latter (N-terminus, side chain of either or of both lysine residues), and (c) length of the spacer between the imidazolium moiety and the peptide's N-terminus. To this end, the PP4 sequence was first assembled, according to steps ii and iii in FIG. 1-B, and conveniently protected lysine (Fmoc-Lys(Boc)-OH) or azido-lysine (Fmoc-Lys(N₃)-OH) building blocks were inserted in the respective positions of the sequence, according to the desired site for the subsequent introduction of the imidazolium moiety via “click” copper(I)-catalyzed alkyne-azide cycloaddition (CuAAC). To produce the peptides modified at the N-terminus, the sequence bearing two natural Lys residues was assembled and further elongated through coupling of azido acetic acid (step iv, FIG. 1-B) yielding a 2-carbon (ethyl) spacer between the N-terminal lysine and the imidazolium moiety to be incorporated via CuAAC. This click reaction was next performed on-resin on all precursor azido-peptides, using the desired propargyl-imidazolium IL (step v, FIG. 1-B) and CuAAC conditions previously reported by us (31). After acidolytic cleavage (step vi, FIG. 1-B) and purification of the crude conjugates thus obtained by reverse-phase preparative high performance liquid chromatography (RP-HPLC), all the resulting IL-KTTKS conjugates were isolated in high purity (>95%), and their expected molecular weights confirmed by ESI-IT-MS.

In addition to the target conjugates, the reference cosmeceutical peptide Matrixyl® (C₁₆-KTTKS-OH), its C-terminal carboxamide analogue (C₁₆-KTTKS-NH₂), and the native PP4 (KTTKS) were also assembled by SPPS, following procedures recently reported (32). For the palmitoylated peptides, after the full amino acid sequence of PP4 was assembled, palmitic acid (C₁₆) was coupled. Then, acidolytic cleavage from the solid support delivered the crude peptides that were purified by RP-HPLC. The final peptides were obtained in high purity and their molecular weights confirmed by ESI-IT MS.

### 2. Antibacterial Activity In Vitro

The antimicrobial activity of the IL-KTTKS conjugates was assessed in vitro against reference bacterial strains (American Type Culture Collection, ATCC). The minimal inhibitory concentration (MIC) was determined according to the Clinical and Laboratory Standards Institute (CLSI) guidelines (43) against Gram-positive (S. aureus, E. faecalis) and Gram-negative (E. coli, P. aeruginosa) bacteria. The MIC values obtained are shown in Table 1. Notably, the reference peptides C₁₆-KTTKS-NH₂ and C₁₆-KTTKS-OH were respectively soluble in water and dimethyl sulfoxide (DMSO), but both precipitated when diluted in cation-adjusted Mueller-Hinton broth (MHB2), the culture medium recommended by the CLSI guidelines, which hampered the determination of the MIC values for these reference peptides. Data in Table 1 show, as expected, that the peptide KTTKS alone is devoid of significant antibacterial activity, and that MIC values for the [C₁₆ M1Im][Br] IL are in agreement with those previously reported (44). Interestingly, all conjugates bearing methyl imidazolium (MeIm) units were inactive against the bacterial tested species, even at the highest concentrations used, regardless the number or position of the MeIm moieties in the overall structure. In turn, replacing the methyl substituent in the imidazolium ring by either a tetradecyl (C₁₄) or a hexadecyl (C₁₆) group, led to an improvement in the antibacterial activity, delivering MIC values from 6.45 to 52.6 μg/mL, hence adding antimicrobial activity o the parent KTTKS peptide. Given that KTTK(C₁₆Im)S and C₁₆Im-KTTKS showed the strongest antibacterial activities, and reflect two different conjugation positions, both these peptides were further investigated by determining their MIC against S. epidermidis, S. pyogenes (both Gram-positive) and K. pneumoniae (Gram-negative), chosen due to their abundance in the skin (S. epidermidis) (45), relevance to cSSTI (S. pyogenes) (46-48), and relation to the so-called “ESKAPE” pathogens (K. pneumoniae) (49). The noncovalent mixture of the parent peptide KKTKS and the [C₁₆ M1Im][Br] ionic liquid, presented MIC values comparable to those of [C₁₆ M1Im][Br] alone.

TABLE 1
MIC values (n = 3) in μM (in μg/mL) of the IL-KTTKS conjugates against Gram-negative and Gram-positive bacteria (ATCC reference strains).

| | MIC in μM (in μg/mL) | | | | | | | | | | | | | |
|---|---|---|---|---|---|---|---|---|---|---|---|---|---|---|
| | | | P. | | | | E. | | K. | | S. | | S. | |
| | E. coli | | aeruginosa | | S. aureus | | faecalis | | pneumonia | | epidermidis | | pyogenes | |
| | ATCC | | ATCC | | ATCC | | ATCC | | ATCC | | ATCC | | ATCC | |
| Peptide | 25922 | | 27853 | | 29213 | | 29212 | | 138830 | | 14990 | | 19615 | |
| K(MeIm)TTKS | >1030.1 (731.2) | | | | | | | | ND^{a} | | ND^{a} | | ND^{a} | |
| KTTK(MeIm)S | >954.2 (677.3) | | | | | | | | ND^{a} | | ND^{a} | | ND^{a} | |
| K(MeIm)TTK(MeIm)S | >1245.5 (1067.4) | | | | | | | | ND^{a} | | ND^{a} | | ND^{a} | |
| KTTK(C₁₄Im)S | 29.5 | (26.3) | 58.9 | (52.6)^{b} | 29.5 | (26.3) | 58.9 | (52.6)^{b} | ND^{a} | | ND^{a} | | ND^{a} | |
| KTTK(C₁₆Im)S | 7.0 | (6.45) | 32.5 | (29.9) | 14.0 | (12.9) | 32.5 | (29.9) | 53.8 | (49.5) | 5.4 | (5.0) | 10.9 | (10.0) |
| MeIm-KTTKS | >825.9 (633.4) | | | | | | | | ND^{a} | | ND^{a} | | ND^{a} | |
| C₁₆Im-KTTKS | 14.3 | (14.0) | 28.7 | (28.0) | 14.3 | (14.0) | 28.7 | (28.0) | 27.4 | (26.8) | 9.5 | (9.3) | 18.9 | (18.5) |
| KTTKS | >1820^{e} | | | | | | | | ND^{a} | | ND^{a} | | ND^{a} | |
| [C₁₆M1Im][Br] | 60 | | >240 | | 0.94^{d} | | 0.94^{c} | | 60 | | ND^{a} | | ND^{a} | |
| KTTKS:[C₁₆M1Im][Br] | 60 | | >240 | | 0.94^{d} | | 0.18^{c} | | 60 | | ND^{a} | | ND^{a} | |
| (1:1) | | | | | | | | | | | | | | |
| Ciprofloxacin | 0.012 | (0.004) | 0.18 | (0.06) | 1.5 | (0.5) | 0.38 | (0.125) | 0.75 | (0.25) | 0.75 | (0.25) | 6.04 | (2.0) |

^{a}Not Determined;
^{b}the MBC was 2× the MIC;
^{c}MBC = 15 μM;
^{d}MBC = 30 μM; in all other cases, the MBC was equal to the MIC;
^{e}value from ref (32)

The antibacterial activities of the best couple of conjugates, i.e., C16Im-KTTKS and KTTK(C16Im)S, and of the reference antibiotic ciprofloxacin, were further assessed against MDR clinical isolates of K. pneumoniae (KP010), S. aureus (SA007), and P. aeruginosa (PA004). MIC values thus obtained are displayed in Table 2 and show that both conjugates preserve their antibacterial activity observed against susceptible ATCC bacterial strains. Relevantly, the conjugates were clearly more active than the reference antibiotic ciprofloxacin against the MDR isolates; for instance, the MIC value obtained for C16Im-KTTKS against SA007 is nearly 10-fold higher than that of ciprofloxacin.

TABLE 2
MIC values (n = 3) in μM (in μg/mL)
for C16-Im-KTTKS and KTTK(C16Im)S against MDR
clinical isolates of Gram-positive and Gram-negative bacteria

Peptide

| MDR | C₁₆Im-KTTKS | | KTTK(C₁₆Im)S | Ciprofloxacin | |
|---|---|---|---|---|---|
| KP010 | 37.9 | (37.0) | 21.7 (20.0) | 48.0 | (16.0) |
| PA004 | 18.9 | (18.5) | | 96.0 | (32.0) |
| SA007 | 18.9 | (18.5)^{a} | | 193.0 | (64.0) |

^{a}The MBC was 2× the MIC; In all other cases the MBC was equal to the MIC

The antibacterial activity of C₁₆Im-KTTKS and KTTK(C₁₆Im)S was also assessed in SWF (50) against S. aureus (ATCC 29213), to check if it was preserved in this medium. MIC values were obtained in both SWF and MHB media in three independent experiments run in triplicates (Table 3), and indicated that the antibacterial activity in SWF was the same as that in MHB for C₁₆Im-KTTKS, and decreased for KTTK(C₁₆Im)S displaying a MIC twice as low.

TABLE 3
MIC [MBC] values (n = 3) in μg/mL
for C16Im-KTTKS and KTTK(C16Im)S against S. aureus
(ATCC 29213) in MHB and SWF.

MIC in μg/mL [MBC]

| Peptide | MHB | | SWF | |
|---|---|---|---|---|
| C₁₆Im-KTTKS | 16-32 | [32-64] | 16-32 | [32->64] |
| KTTK(C₁₆Im)S | 32 | [64-128] | 64-128 | [128->128] |

### 3. Antifungal Activity In Vitro

The antifungal activity of the best couple of IL-KTTKS conjugates, their parent building blocks, and respective noncovalent 1:1 mixture, were all assessed against three species of Candida, namely, Candida albicans (ATCC 90028), Candida glabrata (ATCC 90030), and Candida parapsilosis (ATCC 22019). The MIC values were determined according to the European Committee on Antimicrobial Susceptibility Testing (EUCAST) protocol (51-53) and are shown on Table 4. Both conjugates, KTTK(C₁₆Im)S and C₁₆Im-KTTKS, were equally active against all Candida spp., with MIC values ranging from 2.4 to 5.4 μM. Both peptides were twice more active against C. parapsilosis than against the other two Candida species. Relevantly, the noncovalent mixture KTTKS:[C₁₆M1Im][Br] (1:1) showed a potent activity against all Candida spp., being equipotent to the parent IL alone, and both seven times more active than the reference antifungal drug fluconazole.

TABLE 4
MIC values (n = 2) in μM (in μg/mL) for the
best performing conjugates, their parent building blocks,
and respective 1:1 noncovalent mixture on ATCC Candida spp.

| | MIC in μM (in μg/mL) | | | | |
|---|---|---|---|---|---|
| | C. albicans | C. glabrata | | | |
| | ATCC | ATCC | | C. parapsilosis | |
| Peptide | 90028 | 90030 | | ATCC 22019 | |
| KTTK(C₁₆Im)S | 5.4 (5.0) | 5.4 | (5.0) | 2.7 | (2.5) |
| C₁₆Im-KTTKS | 4.7 (4.6) | 4.7 | (4.6) | 2.4 | (2.3) |
| KTTKS | >60 | >60 | | >60 | |
| [C₁₆M1Im][Br] | 0.93 | 0.93 | | 0.93 | |
| KTTKS:[C₁₆M1Im][Br] | 0.93 | 0.93 | | 0.93 | |
| (1:1) | | | | | |
| Fluconazole | 1.6 (0.5) | 26 | (8) | 6.5 | (2) |

### 4. Toxicity to HFF-1 and HaCaT Cells

The cytotoxicity of KTTK(C₁₆Im)S and C₁₆Im-KTTKS conjugates was assessed on human foreskin fibroblasts (HFF-1) and human immortalized keratinocytes (HaCaT). The results shown in Table 5, are expressed as the conjugate concentration causing a 50% cell growth inhibition (IC₅₀). As expected from previous reports (54), both the parent peptide sequence KTTKS and the derived reference cosmeceutical Matrixyl® (C₁₆-KTTKS-OH) did not show relevant toxicity against the cell lines tested, at up to 100 μM. In turn, the parent IL [C₁₆ M1Im][Br] and its covalent equimolar mixture with PP4, KTTKS:[C₁₆ M1Im][Br] (1:1), were significantly toxic. Interestingly, covalent conjugation of the peptide to the IL resulted in an intermediate situation, as conjugates were more toxic than the peptide alone, but clearly less toxic than the IL alone or than its noncovalent mixture with the peptide.

### 5. Collagen Production In Vitro

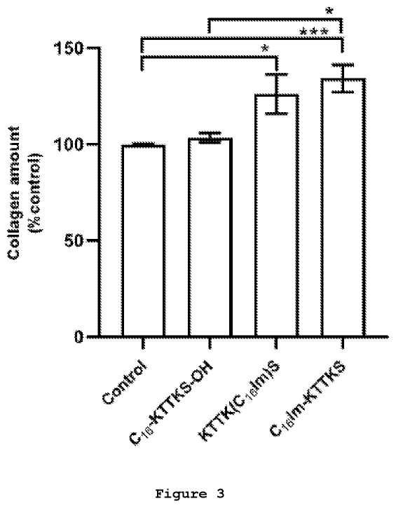

The point of conjugating antimicrobial ILs to a collagenesis-inducing peptide was to afford a simple construct able to exert a dual antimicrobial and skin rebuilding action. Therefore, the two best IL-KTTKS conjugates were further tested for their ability to promote collagen production by human dermal fibroblasts (HDF) in vitro. This was assessed using the Sircol® kit assay, whereby the amount of newly formed collagen in the ECM that is deposited in the microwell-plated cell cultures is solubilized in an acidic medium and next quantified through a collagen standard curve according to the Sircol® kit assay procedure (55). Assays were conducted in different conditions for comparison, namely, in the presence of the reference cosmeceutical Matrixyl® (positive control—C₁₆-KTTKS-OH), of the test conjugates KTTK(C₁₆Im)S and C₁₆Im-KTTKS, and in the absence of any peptide (negative control). Data presented in FIG. 3 show that both conjugates induce HDF cells to produce more collagen, as compared to the negative control. No significant difference was observed between both conjugates or between KTTK(C₁₆Im)S conjugate and reference Matrixyl®, demonstrating that the ability of Matrixyl® to induce collagenesis is not affected by the introduction of the imidazolium IL at the Lys side chain of the peptide sequence.

This description is of course not in any way restricted to the forms of implementation presented herein and any person with an average knowledge of the area can provide many possibilities for modification thereof without departing from the general idea as defined by the claims. The preferred forms of implementation described above can obviously be combined with each other. The following claims further define the preferred forms of implementation.

### SEQUENCE LISTING

SEQ ID NO 1=PP4
SEQ ID NO 2=PKEK
SEQ ID NO 3=GEKG
SEQ ID NO 4=tetrapeptide-3
SEQ ID NO 5=pentapeptide-3
SEQ ID NO 6=pentapeptide-18
SEQ ID NO 7=hexapeptide-11
SEQ ID NO 8=acetyl hexapeptide-3
SEQ ID NO 9=hexapeptide-10
SEQ ID NO 10=hexapeptide-12
SEQ ID NO 11=acetyl octapeptide-1
SEQ ID NO 12=SA1-III
SEQ ID NO 13=KVK
SEQ ID NO 14=GHK
SEQ ID NO 15=copper tripeptide-1=Cu(II)-GHK
SEQ ID NO 16=manganese tripeptide-1=Mn(II)-GHK
SEQ ID NO 17=palmitoyl tripeptide-1=C16-GHK (palmitoyl from palmitic acid, CH3—(CH2)14—COOH)
SEQ ID NO 18=palmitoyl tripeptide-5=C16-KVK
SEQ ID NO 19=palmitoyl hexapeptide-12=C16-VGVAPG
SEQ ID NO 20=lipospondin=elaidyl-KFK (elaidyl from elaidic acid, HOOC—(CH₂)₇—CH═CH—(CH₂)₇—CH3)

### BIBLIOGRAPHY

1. Egorova, K. S.; Ananikov, V. P. Fundamental importance of ionic interactions in the liquid phase: A review of recent studies of ionic liquids in biomedical and pharmaceutical applications. J. Mol. Liq. 2018, 272, 271-300.
2. Torimoto, T.; Tsuda, T.; Okazaki, K.-I.; Kuwabata, S. New Frontiers in Materials Science Opened by Ionic Liquids. Adv. Mater. 2010, 22, 1196-1221.
3. Roosen, C.; Muller, P.; Greiner, L. Ionic liquids in biotechnology: Applications and perspectives for biotransformations. Appl. Microbiol. Biotechnol. 2008, 81, 607-614.
4. Egorova, K. S.; Gordeev, E. G.; Ananikov, V. P. Biological Activity of Ionic Liquids and Their Application in Pharmaceutics and Medicine. Chem. Rev. 2017, 117, 7132-7189.
5. Ferraz, R.; Branco, L. C.; Prudêncio, C.; Noronha, J. P.; Petrovski, Z. Ionic liquids as active pharmaceutical ingredients. ChemMedChem 2011, 6, 975-985.
6. Dias, A. R.; Costa-Rodrigues, J.; Fernandes, M. H.; Ferraz, R.; Prudêncio, C. The Anticancer Potential of Ionic Liquids. ChemMedChem 2017, 12, 11-18.
7. Ferraz, R.; Pinheiro, M.; Gomes, A.; Teixeira, C.; Prudêncio, C.; Reis, S.; Gomes, P. Effects of novel triple-stage antimalarial ionic liquids on lipid membrane models. Bioorg. Med. Chem. Lett. 2017, 27, 4190-4193.
8. Pendleton, J. N.; Gilmore, B. F. The antimicrobial potential of ionic liquids: A source of chemical diversity for infection and biofilm control. Int. J. Antimicrob. Agents 2015, 46, 131-139.
9. Venkata Nancharaiah, Y.; Reddy, G. K.; Lalithamanasa, P.; Venugopalan, V. P. The ionic liquid 1-alkyl-3-methylimidazolium demonstrates comparable antimicrobial and antibiofilm behavior to a cationic surfactant. Biofouling 2012, 28, 1141-1149.
10. Sidat, Z.; Marimuthu, T.; Kumar, P.; du Toit, L. C.; Kondiah, P. P. D.; Choonara, Y. E.; Pillay, V. Ionic Liquids as Potential and Synergistic Permeation Enhancers for Transdermal Drug Delivery. Pharmaceutics 2019, 11, 96.
11. Zakrewsky, M.; Lovejoy, K. S.; Kern, T. L.; Miller, T. E.; Le, V.; Nagy, A.; Goumas, A. M.; Iyer, R. S.; Del Sesto, R. E.; Koppisch, A. T.; et al. Ionic liquids as a class of materials for transdermal delivery and pathogen neutralization. Proc. Natl. Acad. Sci. USA 2014, 111, 13313-13318.
12. Vos, T. et al. (2020). Global burden of 369 diseases and injuries in 204 countries and territories, 1990-2019: a systematic analysis for the Global Burden of Disease Study 2019. Global H
13. Macedo, G. H. R. V. et al. (2021). Interplay between ESKAPE Pathogens and Immunity in Skin Infections: An Overview of the Major Determinants of Virulence and Antibiotic Resistance. Pathogens, 10(2), article 148. https://doi.org/10.3390/pathogens10020148
14. Kalan L., et al. (2016). Redefining the Chronic-Wound Microbiome: Fungal Communities Are Prevalent, Dynamic, and Associated with Delayed Healing. mBio, 7(5):e01058-16. https://doi.org/10.1128/mBio.01058-16.
15. Wang, R., et al. (2020). Managing chronic wounds during novel coronavirus pneumonia outbreak. Burns & Trauma, 8, tkaa016. https://doi.org/10.1093/burnst/tkaa016.
16. Leong H N, Kurup A, Tan M Y, Kwa A L H, Liau K H, Wilcox M H. 2018. Management of complicated skin and soft tissue infections with a special focus on the role of newer antibiotics. Infect Drug Resist 11:1959-1974.
17. Golan Y. 2019. Current Treatment Options for Acute Skin and Skin-structure Infections. Clinical infectious diseases: an official publication of the Infectious Diseases Society of America 68:S206-S212.
18. Esposito S, Ascione T, Pagliano P. 2019. Management of bacterial skin and skin structure infections with polymicrobial etiology. Expert Rev Anti Infect Ther 17:17-25.
19. Kalan L, Grice E A. 2018. Fungi in the Wound Microbiome. Adv Wound Care 7:247-255.
20. Beyene R T, Derryberry S L, Jr., Barbul A. 2020. The Effect of Comorbidities on Wound Healing. Surg Clin North Am 100:695-705.
21. Gonzalez A C d O, Costa T F, Andrade Z d A, Medrado A R A P. 2016. Wound healing—A literature review. Anais brasileiros de dermatologia 91:614-620.
22. Chattopadhyay S, Raines R T. 2014. Review collagen-based biomaterials for wound healing. Biopolymers 101:821-833.
23. Banerjee P, Suguna L, Shanthi C. 2015. Wound healing activity of a collagen-derived cryptic peptide. Amino Acids 47:317-328.
24. Sato K, Jimi T T, 2020. Asai S. Collagen-Derived Di-Peptide, Prolylhydroxyproline (Pro-Hyp): A New Low Molecular Weight Growth-Initiating Factor for Specific Fibroblasts Associated With Wound Healing. Front Cell Dev Biol 8.
25. Sivaraman K, Shanthi C. 2018. Matrikines for therapeutic and biomedical applications. Life Sci 214:22-33.
26. Maquart F X, Bellon G, Pasco S, Monboisse J C. 2005. Matrikines in the regulation of extracellular matrix degradation. Biochimie 87:353-360.
27. ABU SAMAH N H, HEARD C M. 2011. Topically applied KTTKS: a review. Int J Cosmet Sci 33:483-490.
28. Katayama K, Armendariz-Borunda J, Raghow R, Kang A H, Seyer J M. 1993. A pentapeptide from type I procollagen promotes extracellular matrix production. J Biol Chem 268:9941-4.
29. Aldag C, Nogueira Teixeira D, Leventhal P S. 2016. Skin rejuvenation using cosmetic products containing growth factors, cytokines, and matrikines: a review of the literature. Clin Cosmet Investig Dermatol 9:411-419.
30. Robinson L R, Fitzgerald N C, Doughty D G, Dawes N C, Berge C A, Bissett D L. 2005. Topical palmitoyl pentapeptide provides improvement in photoaged human facial skin. Int J Cosmet Sci 27:155-160.
31. Gomes A, Bessa L J, Correia P, Fernandes I, Ferraz R, Gameiro P, Teixeira C, Gomes P. 2020. “Clicking” an Ionic Liquid to a Potent Antimicrobial Peptide: On the Route towards Improved Stability. Int J Mol Sci 21:1-111
32. Gomes A, Bessa L J, Fernandes I, Ferraz R, Mateus N, Gameiro P, Teixeira C, Gomes P. 2019. Turning a Collagenesis-Inducing Peptide Into a Potent Antibacterial and Antibiofilm Agent Against Multidrug-Resistant Gram-Negative Bacteria. Frontiers in Microbiology 10:1-11.
33. Reddy G K K, Nancharaiah Y V. 2020. Alkylimidazolium Ionic Liquids as Antifungal Alternatives: Antibiofilm Activity Against Candida albicans and Underlying Mechanism of Action. Frontiers in microbiology 11:730-730.
34. Forero Doria O, Castro R, Gutierrez M, Gonzalez Valenzuela D, Santos L, Ramirez D, Guzman L. 2018. Novel Alkylimidazolium Ionic Liquids as an Antibacterial Alternative to Pathogens of the Skin and Soft Tissue Infections. Molecules (Basel, Switzerland) 23:2354.
35. Zhang D, Wang H-J, Cui X-M, Wang C-X. 2016. Evaluations of imidazolium ionic liquids as novel skin permeation enhancers for drug transdermal delivery. Pharm Dev Technol 22:1-10.
36. Gomes A, Aguiar L, Ferraz R, Teixeira C, Gomes P. 2021. The Emerging Role of Ionic Liquid-Based Approaches for Enhanced Skin Permeation of Bioactive Molecules: A Snapshot of the Past Couple of Years. International Journal of Molecular Sciences 22.
37. Colonna M, Berti C, Binassi E, Fiorini M, Sullalti S, Acquasanta F, Vannini M, Di Gioia D, Aloisio I, Karanam S, Brunelle D J. 2012. Synthesis and characterization of imidazolium telechelic poly(butylene terephthalate) for antimicrobial applications. React Funct Polym 72:133-141.
38. Gomes, A. et al. 2021. Disclosure of a Promising Lead to Tackle Complicated Skin and Skin Structure Infections: Antimicrobial and Antibiofilm Actions of Peptide PP4-3.1 Vol. 13, no. 1962 p. 1962.57.
39. Jeong, W., et al. 2021. Recent advances in transdermal drug delivery systems: a review. Biomaterials Research volume 25.
40. Kawada-Matsuo, M. et al. 2021. Antibacterial Peptides Resistance in Staphylococcus aureus: Various Mechanisms and the Association with Pathogenicity. Genes 2021, 12(10), 1527.
41. Monti, D. et al. (2018). Influence of a Combination of Chemical Enhancers and Iontophoresis on In Vitro Transungual Permeation of Nystatin. AAPS PharmSciTech, 19(4), 1574-1581.
42. Hu Q, Deng Y, Yuan Q, Ling Y, Tang H. 2014. Polypeptide ionic liquid: Synthesis, characterization, and application in single-walled carbon nanotube dispersion. J Polym Sci Part A: Polym Chem 52:149-153.
43. Patel J B. 2012. Methods for Dilution Antimicrobial Susceptibility Tests for Bacteria that Grow Aerobically; Approved Standard M7-A9; Clinical and Laboratory Standards Institute: Wayne, PA, USA.
44. ostleb F, Stefanik D, Seifert H, Giernoth R. 2013. BIOnic Liquids: Imidazolium-based Ionic Liquids with Antimicrobial Activity. Z Naturforsch B 68:1123-1128.
45. Büttner H, Mack D, Rohde H. 2015. Structural basis of Staphylococcus epidermidis biofilm formation: mechanisms and molecular interactions. Frontiers in cellular and infection microbiology 5:1-14.
46. Sadeghpour Heravi F, Zakrzewski M, Vickery K, G Armstrong D, Hu H. 2019. Bacterial Diversity of Diabetic Foot Ulcers: Current Status and Future Prospectives. Journal of clinical medicine 8:1935.
47. Walker Mark J, Barnett Timothy C, McArthur Jason D, Cole Jason N, Gillen Christine M, Henningham A, Sriprakash K S, Sanderson-Smith Martina L, Nizet V. 2014. Disease Manifestations and Pathogenic Mechanisms of Group A Streptococcus. Clinical Microbiology Reviews 27:264-301.
48. Pato C, Melo-Cristino J, Ramirez M, Friães A, TPGftSoSI, Vaz T, Gião M, Ferreira R, Silva A C, Costa H, Silva M F, Afonso M A, Domingos A, Marrão G, Grossinho J, Lopes P, Lameirão A, Abreu G, Selaru A, Marques H, Tomaz M, Mota P, Ramos M H, Castro A P, Fonseca F, Canhoto N, Afonso T, Pina T, Peres H, Chantre O, Marques J, Marcelo C, Peres I, Lourenço I, Pinto M, Monteiro L, Lito L M, Toscano C, Ana Pessanha M, Ramalheira E, Diaz R, Ferreira S, Roxo I C, Castro A P, Ribeiro G, Tomé R, Pontes C, Boaventura L, Chaves C, Reis T, et al. 2018. Streptococcus pyogenes Causing Skin and Soft Tissue Infections Are Enriched in the Recently Emerged emm89 Clade 3 and Are Not Associated With Abrogation of CovRS. Frontiers in Microbiology 9.
49. Mulani M S, Kamble E E, Kumkar S N, Tawre M S, Pardesi K R. 2019. Emerging Strategies to Combat ESKAPE Pathogens in the Era of Antimicrobial Resistance: A Review. Frontiers in microbiology 10:539-539.
50. Price B L, Lovering A M, Bowling F L, Dobson C B. 2016. Development of a Novel Collagen Wound Model To Simulate the Activity and Distribution of Antimicrobials in Soft Tissue during Diabetic Foot Infection. Antimicrobial agents and chemotherapy 60:6880-6889.
51. EUCAST. 2020. The European Committee on Antimicrobial Susceptibility Testing.
52. EUCAST. 2020. The European Committee on Antimicrobial Susceptibility Testing.
53. EUCAST. 2020. The European Committee on Antimicrobial Susceptibility Testing.
54. Tałałaj U, Uścinowicz P, Bruzgo I, Surażyński A, Zaręba I, Markowska A. 2019. The Effects of a Novel Series of KTTKS Analogues on Cytotoxicity and Proteolytic Activity. Molecules (Basel, Switzerland) 24:3698.
55. Anonymous. Sircol Collagen Assay Kit. https://www.biocolor.co.uk/product/sircol-soluble-collagen-assay/. Accessed 22 Sep. 2021.

1. A peptide-ionic liquid conjugate for the prevention and/or treatment of skin disorders, wherein the peptide-ionic liquid conjugate comprises a cosmeceutical peptide comprising up to 10 amino acids and at least one ionic liquid comprising saturated or unsaturated hydrocarbon chain substituents from C1 to C18.

2. The peptide-ionic liquid conjugate according to claim 1, wherein the cosmeceutical peptide is selected from the group consisting of sequences SEQ ID NO: 1 to 20.

3. The peptide-ionic liquid conjugate according to claim 1, wherein the at least one ionic liquid is selected from a pyridinium, an imidazolium, a phosphonium, or a cholinium ionic liquid.

4. The peptide-ionic liquid conjugate according to claim 1, wherein the conjugation between the cosmeceutical peptide and at least one ionic liquid occurs on the N-terminus of an amino acid of the cosmeceutical peptide.

5. The peptide-ionic liquid conjugate according to claim 1, wherein the conjugation between the cosmeceutical peptide and at least one ionic liquid occurs on a side chain of an amino acid of the cosmeceutical peptide.

6. The peptide-ionic liquid conjugate according to claim 1, wherein the conjugation between the cosmeceutical peptide and the ionic liquids occurs on the N-terminus of an amino acid and on a side chain of an amino acid of the cosmeceutical peptide.

7. The peptide-ionic liquid conjugate according to claim 1, wherein the skin disorders are severe non-healing skin ulcerations, milder bacterial or fungal skin infections, auto-immune/inflammatory disorders, aging and cancer skin disorders, either in absence or presence of secondary infection, treatment of post-surgical wounds, common injuries or burns.

8. A topical composition comprising at least one peptide-ionic liquid conjugate according to claim 1.

9. The topical composition according to claim 8, further comprising nanoparticles, solid lipid nanoparticles, nanostructured lipid carriers including liposomes or polymeric nanoparticles, and/or at least one hydrogel including polysaccharide-based hydrogels or poly (lactic-co-glycolic acid)-based hydrogels.

10. A method of treating skin a disorder comprising applying the topical composition according to claim 8 to the skin disorder.

11. The method according to claim 10, wherein the skin disorder is severe non-healing skin ulcerations, milder bacterial or fungal skin infections, auto-immune/inflammatory disorders, aging and cancer skin disorders, either in absence or presence of secondary infection, treatment of post-surgical wounds, common injuries or burns.

12. The peptide-ionic liquid conjugate according to claim 7, wherein the severe non-healing skin ulcerations are diabetic foot ulcers, pressure ulcers, venous leg ulcers; the milder bacterial or fungal skin infections are atopic dermatitis, candidiasis, cellulitis, dermatophytoses, erysipelas, folliculitis, impetigo, rosacea; the auto-immune/inflammatory disorders, aging and cancer skin disorders are melanoma, non-melanoma skin cancers, psoriasis, cutaneous lupus erythematosus, impetigo, skin aging, and melanoma.

13. The method according to claim 11, wherein the severe non-healing skin ulcerations are-diabetic foot ulcers, pressure ulcers, venous leg ulcers; the milder bacterial or fungal skin infections are acne, atopic dermatitis, candidiasis, cellulitis, dermatophytoses, erysipelas, folliculitis, impetigo, rosacea; the auto-immune/inflammatory disorders, aging and cancer skin disorders are melanoma, non-melanoma skin cancers, psoriasis, cutaneous lupus erythematosus, impetigo, skin aging, and melanoma.


SEQUENCE LISTING (12 sequences)

SEQ ID NO: 1 - protein, 5 aa, synthetic construct
KTTKS

SEQ ID NO: 2 - protein, 4 aa, synthetic construct
PKEK

SEQ ID NO: 3 - protein, 4 aa, synthetic construct
GEKG

SEQ ID NO: 4 - protein, 4 aa, synthetic construct
KGHK

SEQ ID NO: 5 - protein, 5 aa, synthetic construct
GPRPA

SEQ ID NO: 6 - protein, 5 aa, synthetic construct
YAGFL

SEQ ID NO: 7 - protein, 6 aa, synthetic construct
FVAPFP

SEQ ID NO: 8 - protein, 6 aa, synthetic construct
EEMQRR

SEQ ID NO: 9 - protein, 6 aa, synthetic construct
SIKVAV

SEQ ID NO: 10 - protein, 6 aa, synthetic construct
VGVAPG

SEQ ID NO: 11 - protein, 8 aa, synthetic construct
EEMQRRAD

SEQ ID NO: 12 - protein, 10 aa, synthetic construct
MGKVVNPTQK